SEQUENCE LISTING (6 sequences)

SEQ ID NO: 1 - protein, 28 aa, Baculovirus
KFNRCIKRKVEHRVKKRPPTWRHNVRAK

SEQ ID NO: 2 - protein, 13 aa, Artificial sequence
  Complement inhibitor peptide
ICVVQDWGHHRCT

SEQ ID NO: 3 - DNA, 44 bp, Homo sapiens
ctgcagcaacattggagctacggcttgcgcccgggcgcggtacc

SEQ ID NO: 4 - protein, 14 aa, Homo sapiens
LQQHWSYGLRPGAV

SEQ ID NO: 5 - DNA, 53 bp, Homo sapiens
ctgcaggaattgtacgaaaacaaaccgcgccgcccgtacattttggcggtacc

SEQ ID NO: 6 - protein, 17 aa, Homo sapiens
LQELYENKPRRPYILAV

(19) United States
(12) Patent Application Publication
(10) Pub. No.: US 2009/0136451 A1
(43) Pub. Date: May 28, 2009

(54) Humanised Baculovirus

(76) Inventors: Norman Maitland, York (GB); Lindsay Stanbridge, York (GB)

Correspondence Address: MARSHALL, GERSTEIN & BORUN LLP, 233 SOUTH WACKER DRIVE, 6300 SEARS TOWER, CHICAGO, IL, 60606-6357, US

(21) Appl. No.: 11/720,068

(22) PCT Filed: Nov. 22, 2005
(86) PCT No.: PCT/GB2005/004465

§ 371 Date: Jan. 8, 2008

(30) Foreign Application Priority Data
Nov. 23, 2004 (GB) 0425739.0

(51) Int. Cl. A61K 35/76; C12N 7/01; A61P 35/00

(52) U.S. Cl. 424/93.2; 435/235.1

(57) ABSTRACT

We describe a modified baculovirus that has increased specific cell targeting and decreased non-specific targeting by mutation of a heparin sulphate binding motif.

The invention relates to a modified baculovirus that has increased specific cell targeting and decreased non-specific targeting.

Gene therapy involves the transfer, and optionally the stable insertion, of new genetic information into cells for the therapeutic treatment of disease. The main issues with respect to gene therapy relate to the efficient targeting of nucleic acid to cells and the establishment of high level transgene expression in selected tissues. A number of methodologies have been developed which purport to facilitate either or both of these requirements. For example, U.S. Pat. No. 6,043,339 disclose the use of signal peptides which when fused to a nucleic acid can facilitate the translocation of the linked nucleic acid across cell membranes. U.S. Pat. No. 6,083,714 discloses a combined nucleic acid and targeting means which uses the polycation poly-lysine coupled to an integrin receptor thereby targeting cells expressing the integrin. EP1013770 discloses the use of nuclear localisation signals (NLS) coupled to oligonucleotides. The conjugate may be covalently linked to vector DNA and the complex used to transfect cells. The NLS sequence serves to facilitate the passage of the vector DNA across the nuclear membrane thereby targeting gene delivery to the nucleus.

A range of viral based vectors have been used to successfully transfect mammalian cell lines. These include adenovirus, adenovirus-associated virus, papovaviruses and vaccinia virus. These viral based vectors have considerable disadvantages. Adenovirus vectors are well established in gene therapy trials. (Wickham T J, Gene therapy, 7: 110, 2000). However, a major problem appears to be non-selective cytotoxicity, particularly in the liver, and pre-existing immune responses against the virus.

An alternative vector, which has been shown to infect mammalian cells, is the baculovirus. Baculovirus is a rod form virus and therefore limitations to the amount of genetic material inserted into recombinant baculovirus is not as limiting as those imposed by adenovirus capsid. The baculovirus will not express its own genes from insect-specific promoters in human cells. This is an attractive feature since the baculovirus will not provoke an immune response as a consequence of viral gene expression of virally encoded genes. However, insertion of a marker or therapeutic gene under control of a mammalian promoter allows high level expression of the transgene. Unlike the adenovirus vector, baculovirus will not recombine with pre-existing material. Infection with baculovirus will not facilitate the replication of endogenous human viruses, as has been demonstrated with adenovirus vectors. In contrast to many of the other therapeutic viruses, baculoviruses can be grown in a serum free culture media in large quantities. This method of production can be readily scaled up to industrial level and removes the potential hazards of serum contamination of the therapeutic agent with viral and prion agents. Most importantly, unlike all other human viral vectors, there is no pre-existing immune response against baculovirus in humans.

WO03/016540 describes a recombinant baculovirus that includes targeting sequences incorporated into the baculovirus genome which facilitate the delivery of the baculovirus and thereby the therapeutic agent to a specific cell type, for example a prostate cell. The gp64 cell surface protein is modified to include a ligand that allows specific binding and internalisation of the baculovirus to a cell receptor expressed by the cell.

We describe a further modification to the baculovirus disclosed in WO03/016540 that shows reduced non-specific binding to cells, particularly liver cells. We have identified a highly basic region in the baculovirus gp64 protein sequence,

²⁶³kfnrcikrkvehrvkkrpptwrhnvrak²⁹⁰

that is involved in binding to heparin sulphate expressed by mammalian cells, in particular liver cells. Modification to this motif will provide a baculovirus that exhibits reduced non-specific binding.

According to an aspect of the invention there is provided a baculovirus wherein the genome of the virus has been modified to include (i) a nucleic acid molecule that encodes a therapeutic agent; (ii) a nucleic acid molecule that encodes a polypeptide that functions to specifically target the baculovirus to at least one cell type; wherein the baculovirus genome is further modified by addition, deletion or substitution of at least one nucleotide base in a part of a baculovirus gene that encodes an amino acid motif that binds heparin sulphate expressed by a cell.

In a preferred embodiment of the invention said modified baculovirus polypeptide that binds heparin sulphate is gp64.

In a preferred embodiment of the invention gp64 is modified at an amino acid motif comprising the amino acid sequence:

hrvk

In a further preferred embodiment of the invention said gp64 is modified at an amino acid motif comprising the amino acid sequence:

kfnrcikrkvehrvkkrpptwrhnvrak

In a preferred embodiment said baculovirus genome is adapted for eukaryotic gene expression of said nucleic acid molecules.

Typically said adaptation includes, by example and not by way of limitation, the provision of transcription control sequences (promoter sequences) that mediates cell/tissue specific expression. These promoter sequences may be cell/tissue specific, inducible or constitutive.

Promoter is an art recognised term and, for the sake of clarity, includes the following features which are provided by example only, and not by way of limitation. Enhancer elements are cis acting nucleic acid sequences often found 5′ to the transcription initiation site of a gene (enhancers can also be found 3′ to a gene sequence or even located in intronic sequences and is therefore position independent). Enhancers function to increase the rate of transcription of the gene to which the enhancer is linked. Enhancer activity is responsive to trans acting transcription factors (polypeptides) which have been shown to bind specifically to enhancer elements. The binding/activity of transcription factors (please see Eukaryotic Transcription Factors, by David S Latchman, Academic Press Ltd, San Diego) is responsive to a number of environmental cues.

Promoter elements also include so called TATA box and RNA polymerase initiation selection (RIS) sequences which function to select a site of transcription initiation. These sequences also bind polypeptides which function, inter alia, to facilitate transcription initiation selection by RNA polymerase.

Adaptations also include the provision of selectable markers and autonomous replication sequences which both facilitate the maintenance of said vector in either the eukaryotic cell or prokaryotic host.

Adaptations which facilitate the expression of baculovirus encoded genes include the provision of transcription termination/polyadenylation sequences. This also includes the provision of internal ribosome entry sites (IRES) which function to maximise expression of baculovirus encoded genes arranged in bicistronic or multi-cistronic expression cassettes.

These adaptations are well known in the art. There is a significant amount of published literature with respect to expression vector construction and recombinant DNA techniques in general. Please see, Sambrook et al (1989) Molecular Cloning: A Laboratory Manual, Cold Spring Harbour Laboratory, Cold Spring Harbour, N.Y. and references therein; Marston, F (1987) DNA Cloning Techniques: A Practical Approach Vol III IRL Press, Oxford UK; DNA Cloning: F M Ausubel et al, Current Protocols in Molecular Biology, John Wiley & Sons, Inc. (1994).

In a preferred embodiment of the invention said eukaryotic expression is through the provision of cancer cell specific promoter elements. Preferably, said promoters are active in prostate cancer cells.

More preferably the promoter elements are selected from the group as represented in Table 1.

In a preferred embodiment of the invention said therapeutic agent is a polypeptide.

Preferably said polypeptide is a tumour suppressor polypeptide selected from the following group represented in Table 2.

In a further preferred embodiment of the invention said polypeptide is an antigenic polypeptide.

Preferably a tumour rejection antigen precursor selected from the following families represented in Table 3.

In a further preferred embodiment said polypeptide is a prostate tumour rejection antigen.

In a further preferred embodiment of the invention said polypeptide is a cytotoxic polypeptide. For example pseudomonas exotoxin, ricin toxin, diphtheria toxin (Genbank acc.#: A04646).

In a yet further preferred embodiment of the invention said polypeptide is a polypeptide which induces cell-cycle arrest.

Preferably said cell-cycle arrest polypeptide is selected from the group represented in Table 4.

In a further preferred embodiment of the invention said therapeutic polypeptide is a pharmaceutically active polypeptide. Preferably said polypeptide is a cytokine.

Preferably said cytokine is selected from the group represented in Table 5.

In a yet further preferred embodiment of the invention said polypeptide is an antibody, or active binding fragment thereof, for example a Fab fragment.

Antibodies or immunoglobulins (Ig) are a class of structurally related proteins consisting of two pairs of polypeptide chains, one pair of light (L) (low molecular weight) chain (κ or λ), and one pair of heavy (H) chains (γ, α, λ, δ and ε), all four linked together by disulphide bonds. Both H and L chains have regions that contribute to the binding of antigen and that are highly variable from one Ig molecule to another. In addition, H and L chains contain regions that are non-variable or constant. The L chains consist of two domains. The carboxy-terminal domain is essentially identical among L chains of a given type and is referred to as the “constant” (C) region. The amino terminal domain varies from L chain to L chain and contributes to the binding site of the antibody. Because of its variability, it is referred to as the “variable” (V) region. The variable region contains complementarity determining regions or CDR's which form an antigen binding pocket. The binding pockets comprise H and L variable regions which contribute to antigen recognition.

It is possible to create single variable regions, so called single chain antibody variable region fragments (scFv's). If a hybridoma exists for a specific monoclonal antibody then it is possible to isolate scFvs from mRNA extracted from said hybridoma via RT PCR.

Alternatively, phage display screening can be undertaken to identify clones expressing scFvs. scFvs are engineered antibody fragments composed of a variable region of the heavy chain and a variable region of the light chain which are coupled via a linker sequence, see Adams and Schier (1999) Journal of Immunological Methods 249-260.

Alternatively said fragments are “domain antibody fragments”. Domain antibodies are the smallest binding part of an antibody (approximately 13 kDa). Examples of this technology is disclosed in U.S. Pat. No. 6,248,516, U.S. Pat. No. 6,291,158, U.S. Pat. No. 6,127,197 and EP0368684 which are all incorporated by reference in their entirety.

In a preferred method of the invention said antibody fragment is a single chain antibody variable region fragment.

In a further preferred embodiment of the invention said antibody is a humanised or chimeric antibody.

A chimeric antibody is produced by recombinant methods to contain the variable region of an antibody with an invariant or constant region of a human antibody. A humanised antibody is produced by recombinant methods to combine the complementarity determining regions (CDRs) of an antibody with both the constant (C) regions and the framework regions from the variable (V) regions of a human antibody.

Chimeric antibodies are recombinant antibodies in which all of the V-regions of a mouse or rat antibody are combined with human antibody C-regions. Humanised antibodies are recombinant hybrid antibodies which fuse the complimentarily determining regions from a rodent antibody V-region with the framework regions from the human antibody V-regions. The C-regions from the human antibody are also used. The complimentarily determining regions (CDRs) are the regions within the N-terminal domain of both the heavy and light chain of the antibody to where the majority of the variation of the V-region is restricted. These regions form loops at the surface of the antibody molecule. These loops provide the binding surface between the antibody and antigen.

Antibodies from non-human animals provoke an immune response to the foreign antibody and its removal from the circulation. Both chimeric and humanised antibodies have reduced antigenicity when injected to a human subject because there is a reduced amount of rodent (i.e. foreign) antibody within the recombinant hybrid antibody, while the human antibody regions do not elicit an immune response. This results in a weaker immune response and a decrease in the clearance of the antibody. This is clearly desirable when using therapeutic antibodies in the treatment of human diseases. Humanised antibodies are designed to have less “foreign” antibody regions and are therefore thought to be less immunogenic than chimeric antibodies.

In a yet still further preferred embodiment of the invention said polypeptide is a polypeptide which induces apoptosis.

Preferably said apoptosis inducing polypeptide is represented in Table 6.

In a yet still further preferred embodiment of the invention said polypeptide is a pro-drug activating polypeptide.

Preferably said prodrug activating polypeptide is represented in Table 7.

In a still further preferred embodiment of the invention said polypeptide has anti-angiogenic activity. For example angiostatin, Tie2 (Genbank acc. no: AF451865), endostatin (Genbank acc. no: NM130445).

In a further preferred embodiment of the invention said therapeutic agent is an antisense nucleic acid molecule.

As used herein, the term “antisense nucleic acid molecule” or “antisense” describes a nucleic acid which hybridizes under physiological conditions to DNA comprising a particular gene or to an mRNA transcript of that gene and thereby, inhibits the transcription of that gene and/or the translation of that mRNA. The antisense molecules are designed so as to interfere with transcription or translation of a target gene upon hybridization with the target gene. Those skilled in the art will recognize that the exact length of the antisense nucleic acid and its degree of complementarity with its target will depend upon the specific target selected, including the sequence of the target and the particular bases, which comprise that sequence.

It is preferred that the antisense nucleic acid be constructed and arranged so as to bind selectively with the target under physiological conditions, i.e., to hybridize substantially more to the target sequence than to any other sequence in the target cell under physiological conditions.

Although nucleic acids may be chosen which are antisense to any region of the gene or mRNA transcripts, in preferred embodiments the antisense nucleic acid correspond to N-terminal or 5′ upstream sites such as translation initiation, transcription initiation or promoter sites. In addition, 3′-untranslated regions may be targeted. The 3′-untranslated regions are known to contain cis acting sequences which act as binding sites for proteins involved in stabilising mRNA molecules. These cis acting sites often form hair-loop structures which function to bind said stabilising proteins. A well known example of this form of stability regulation is shown by histone mRNA's, the abundance of which is controlled, at least partially, post-transcriptionally.

The present invention, thus, contemplates a baculovirus genome which has been modified by incorporation of an antisense nucleic acid to a specific target sequence, for example a target sequence encoding a cell-cycle regulatory gene, (eg p21 (Genbank acc.#: NM_{—}078467, c-myc (Genbank acc.#: D10493 and D90467), cyclin dependent kinase inhibitors, p16 (Genbank acc.#: NM058196), p15 (Genbank acc.#: BC002010), p18 (mouse sequence Genbank acc.#: BC027026), or p19 (Genbank acc.#: NM_{—}079421) and apoptosis inhibitors such as caveolin.

In a further preferred embodiment of the invention said therapeutic agent is a double stranded RNA molecule. In this embodiment the baculovirus genome would include a nucleic acid molecule under the control of a first promoter positioned upstream (ie 5′ of the nucleic acid molecule) and a second promoter positioned downstream (ie 3′ of the nucleic acid molecule). The orientation of the promoters being such that both sense and antisense nucleic acid molecules are produced.

A technique to specifically ablate gene function is through the introduction of double stranded RNA, also referred to as inhibitory RNA (RNAi), into a cell which results in the destruction of mRNA complementary to the sequence included in the RNAi molecule. The RNAi molecule comprises two complementary strands of RNA (a sense strand and an antisense strand) annealed to each other to form a double stranded RNA molecule. The RNAi molecule is typically derived from exonic or coding sequence of the gene which is to be ablated. Alternatively said RNAi molecule is derived from intronic sequences or the 5′ and/or 3′ non-coding sequences which flank coding/exon sequences of genes. Recent studies suggest that RNAi molecules ranging from 100-1000 bp derived from coding sequence are effective inhibitors of gene expression. Surprisingly, only a few molecules of RNAi are required to block gene expression which implies the mechanism is catalytic. The site of action appears to be nuclear as little if any RNAi is detectable in the cytoplasm of cells indicating that RNAi exerts its effect during mRNA synthesis or processing.

The exact mechanism of RNAi action is unknown although there are theories to explain this phenomenon. For example, all organisms have evolved protective mechanisms to limit the effects of exogenous gene expression. For example, a virus often causes deleterious effects on the organism it infects. Viral gene expression and/or replication therefore need to be repressed. In addition, the rapid development of genetic transformation and the provision of transgenic plants and animals has led to the realisation that transgenes are also recognised as foreign nucleic acid and subjected to phenomena variously called quelling (Singer and Selker, Curr Top Microbiol Immunol. 1995; 197:165-77), gene silencing (Matzkeand Matzke, Novartis Found Symp. 1998; 214:168-80; discussion 181-6. Review) and co-suppression (Stam et. al., Plant J. 2000; 21(1):27-42.

In a still further preferred embodiment said therapeutic agent is a ribozyme.

A ribozyme is a catalytic RNA which is well known in the art. A ribozyme comprises a catalytic core having flanking sequences adjacent to the sequence which hybridises to the substrate RNA. The simplest catalytic core is an RNA motif known as a hammerhead. Since the discovery of catalytic RNA there has been a desire to design ribozymes which have a targeted gene function such that disease gene mRNA's can be selectively ablated.

In yet a further preferred embodiment of the invention the baculovirus genome includes a nucleic acid molecule which encodes a polypeptide which binds the baculovirus to the cell surface of at least one cell type. Preferably said baculovirus binds the cell surface by a cell specific cell surface receptor.

In a preferred embodiment of the invention said nucleic acid encodes a polypeptide selected from the following group: GnRH (Genbank acc. no: L03380), fibroblast growth factors; insulin and insulin-like growth factors; neurotensin platelet derived growth factor (Genbank acc. no: NM_{—}002609 & NM_{—}006206); somatostatin (Genbank acc. no: BC032625).

In a preferred embodiment of the invention the nucleic acid encoding said polypeptide is inserted into the baculovirus genome at a site which fuses said polypeptide to a baculovirus capsid polypeptide. Preferably the capsid polypeptide is gp64.

Advantageously the fusion of the targeting polypeptide to a capsid polypeptide will result in its presentation at the baculovirus particle surface thereby presenting the baculovirus to said cell type and thereby facilitating cell targeting.

According to a further aspect of the invention there is provided a pharmaceutical composition comprising the baculovirus according to any previous aspect or embodiment of the invention. Preferably said composition is for use in the manufacture of a medicament for the treatment of cancer, ideally prostate cancer.

When administered, the pharmaceutical compositions of the present invention are administered in pharmaceutically acceptable preparations. Such preparations may routinely contain pharmaceutically acceptable concentrations of salt, buffering agents, preservatives, compatible carriers, supplementary immune potentiating agents such as adjuvants and cytokines and optionally other therapeutic agents, such as chemotherapeutic agents.

The therapeutics of the invention can be administered by any conventional route, including injection or by gradual infusion over time. The administration may, for example, be oral, intravenous, intraperitoneal, intramuscular, intracavity, subcutaneous, or transdermal.

The compositions of the invention are administered in effective amounts. An “effective amount” is that amount of a composition that alone, or together with further doses, produces the desired response. In the case of treating a particular disease, such as cancer, the desired response is inhibiting the progression of the disease. This may involve only slowing the progression of the disease temporarily, although more preferably, it involves halting the progression of the disease permanently. This can be monitored by routine diagnostic methods.

Such amounts will depend, of course, on the particular condition being treated, the severity of the condition, the individual patient parameters including age, physical condition, size and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment. It will be understood by those of ordinary skill in the art, however, that a patient may insist upon a lower dose or tolerable dose for medical reasons, psychological reasons or for virtually any other reasons.

The pharmaceutical compositions used in the foregoing methods preferably are sterile and contain an effective amount of vector for producing the desired response in a unit of weight or volume suitable for administration to a patient. The response can, for example, be measured by determining regression of a tumour, decrease of disease symptoms, modulation of apoptosis, etc.

The doses of vector administered to a subject can be chosen in accordance with different parameters, in particular in accordance with the mode of administration used and the state of the subject. Other factors include the desired period of treatment. In the event that a response in a subject is insufficient at the initial doses applied, higher doses (or effectively higher doses by a different, more localized delivery route) may be employed to the extent that patient tolerance permits.

In general, doses of vector of between 1 ng and 0.1 mg generally will be formulated and administered according to standard procedures. Other protocols for the administration of compositions will be known to one of ordinary skill in the art, in which the dose amount, schedule of injections, sites of injections, mode of administration (e.g. intra-tumoral) and the like vary from the foregoing.

Administration of compositions to mammals other than humans, e.g. for testing purposes or veterinary therapeutic purposes, is carried out under substantially the same conditions as described above. A subject, as used herein, is a mammal, preferably a human or dog.

When administered, the pharmaceutical preparations of the invention are applied in pharmaceutically-acceptable amounts and in pharmaceutically-acceptable compositions. The term “pharmaceutically acceptable” means a non-toxic material that does not interfere with the effectiveness of the biological activity of the active ingredients. Such preparations may routinely contain salts, buffering agents, preservatives, compatible carriers, and optionally other therapeutic agents. When used in medicine, the salts should be pharmaceutically acceptable, but non-pharmaceutically acceptable salts may conveniently be used to prepare pharmaceutically-acceptable salts thereof and are not excluded from the scope of the invention. Such pharmacologically and pharmaceutically-acceptable salts include, but are not limited to, those prepared from the following acids: hydrochloric, hydrobromic, sulfuric, nitric, phosphoric, maleic, acetic, salicylic, citric, formic, malonic, succinic, and the like. Also, pharmaceutically-acceptable salts can be prepared as alkaline metal or alkaline earth salts, such as sodium, potassium or calcium salts.

Compositions may be combined, if desired, with a pharmaceutically-acceptable carrier. The term “pharmaceutically-acceptable carrier” as used herein means one or more compatible solid or liquid fillers, diluents or encapsulating substances which are suitable for administration into a human. The term “carrier” denotes an organic or inorganic ingredient, natural or synthetic, with which the active ingredient is combined to facilitate the application. The components of the pharmaceutical compositions also are capable of being co-mingled with the molecules of the present invention, and with each other, in a manner such that there is no interaction which would substantially impair the desired pharmaceutical efficacy.

The pharmaceutical compositions may contain suitable buffering agents, including: acetic acid in a salt; citric acid in a salt; boric acid in a salt; and phosphoric acid in a salt.

The pharmaceutical compositions also may contain, optionally, suitable preservatives, such as: benzalkonium chloride; chlorobutanol; parabens and thimerosal.

The pharmaceutical compositions may conveniently be presented in unit dosage form and may be prepared by any of the methods well-known in the art of pharmacy. All methods include the step of bringing the active agent into association with a carrier which constitutes one or more accessory ingredients. In general, the compositions are prepared by uniformly and intimately bringing the active compound into association with a liquid carrier, a finely divided solid carrier, or both, and then, if necessary, shaping the product.

Compositions suitable for oral administration may be presented as discrete units, such as capsules, tablets, lozenges, each containing a predetermined amount of the active compound. Other compositions include suspensions in aqueous liquids or non-aqueous liquids such as syrup, elixir or an emulsion.

Compositions suitable for parenteral administration conveniently comprise a sterile aqueous or non-aqueous preparation of vector, which is preferably isotonic with the blood of the recipient. This preparation may be formulated according to known methods using suitable dispersing or wetting agents and suspending agents. The sterile injectable preparation also may be a sterile injectable solution or suspension in a non-toxic parenterally-acceptable diluent or solvent, for example, as a solution in 1,3-butane diol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed including synthetic mono- or di-glycerides. In addition, fatty acids such as oleic acid may be used in the preparation of injectables. Carrier formulation suitable for oral, subcutaneous, intravenous, intramuscular, etc. administrations can be found in Remington's Pharmaceutical Sciences, Mack Publishing Co., Easton, Pa.

In a further preferred embodiment of the invention said composition further comprises at least one further therapeutic agent; preferably, a chemotherapeutic agent.

In a preferred embodiment of the invention said composition includes a complement inhibitor. Preferably, the complement inhibitor comprises the amino acid sequence ICVVQDWGHHRCT-NH₂. Preferably said complement inhibitor consists of the amino acid sequence ICVVQDWGHHRCT-NH₂.

In a preferred embodiment of the invention said complement inhibitor is a variant peptide comprising the amino acid sequence ICVVQDWGHHRCT-NH₂ wherein said sequence is modified by addition, deletion or substitution of at least one amino acid residue and further wherein said inhibitor has improved inhibitory activity with respect to C3 complement protein.

The skilled person has means to modify the sequence of complement inhibitors, for example the modification of compstatin is disclosed in Biochemical Society Transactions (2004) volume 32, part 1, p28 which is incorporated by reference in its entirety.

According to a yet further aspect of the invention there is provided a method of treatment comprising the administration of a therapeutically effective amount of the baculovirus according to the invention to a subject in need of treatment. Preferably said subject is human.

In a preferred method of the invention said treatment is cancer, preferably prostate cancer.

As used herein, the term “cancer” refers to cells having the capacity for autonomous growth, i.e., an abnormal state or condition characterized by rapidly proliferating cell growth. The term is meant to include all types of cancerous growths or oncogenic processes, metastatic tissues or malignantly transformed cells, tissues, or organs, irrespective of histopathologic type or stage of invasiveness. The term “cancer” includes malignancies of the various organ systems, such as those affecting, for example, lung, breast, thyroid, lymphoid, gastrointestinal, and genito-urinary tract, as well as adenocarcinomas which include malignancies such as most colon cancers, renal-cell carcinoma, prostate cancer and/or testicular tumours, non-small cell carcinoma of the lung, cancer of the small intestine and cancer of the esophagus. The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. Exemplary carcinomas include those forming from tissue of the cervix, lung, prostate, breast, head and neck, colon and ovary. The term “carcinoma” also includes carcinosarcomas, e.g., which include malignant tumours composed of carcinomatous and sarcomatous tissues. An “adenocarcinoma” refers to a carcinoma derived from glandular tissue or in which the tumor cells form recognizable glandular structures. The term “sarcoma” is art recognized and refers to malignant tumors of mesenchymal derivation.

Throughout the description and claims of this specification, the words “comprise” and “contain” and variations of the words, for example “comprising” and “comprises”, means “including but not limited to”, and is not intended to (and does not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

An embodiment of this invention will now be provided by example only and with reference to the following materials, methods, vectors and figures:

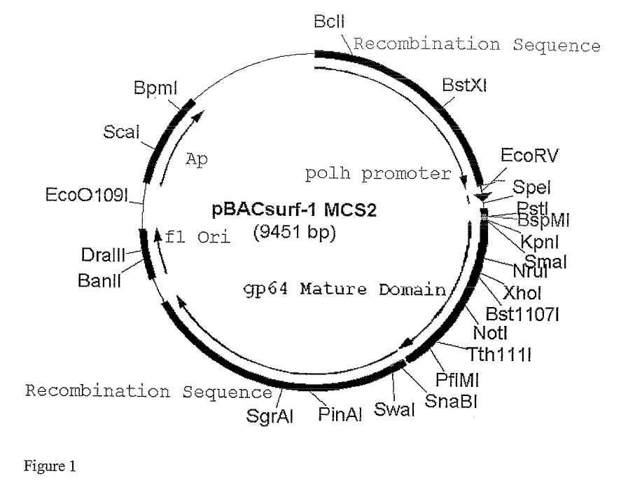

FIG. 1 is baculovirus vector pBAsurf-1 MCS2; and

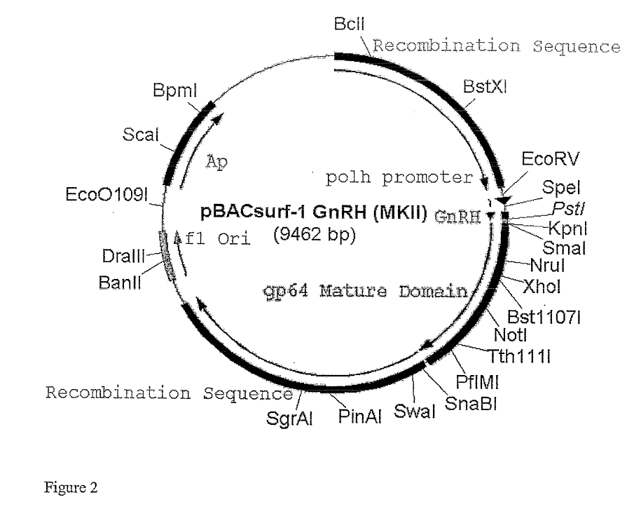

FIG. 2 is baculovirus vector pBAsurf-1 GnRH(MKII); and

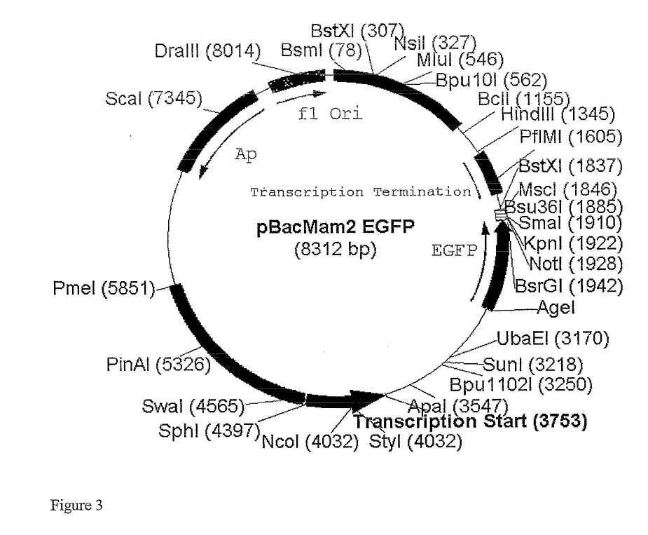

FIG. 3 is baculovirus vector pBacMam2 EGFP.

### MATERIALS AND METHODS

Targeting baculoviruses are generated in two stages (i) by generation of a transfer vector in a bacterial plasmid, which is multiplied in bacteria, and whose DNA sequence in determined to verify the insertion of the recombinant DNA sequence; and (ii) recombination of the transfer vector, via homologous non essential region on either side of the gp64 recombinant, into a multiply cut By genome by cotransfection into recipient insect cells (sf9 or sf21).

An example of the experimental procedure is as follows.

The DNA sequence encoding the minimal peptide required for receptor binding for the GnRH and neurotensin receptors was determined and a DNA oligonucleotides for both strands were chemically synthesised, including PstI and KpnI restriction endonuclease sites to facilitate insertion into the pBACsurf vector. The synthesised oligonucleotides were then ligated into the pBACsurf vector via these restriction endonuclease sites The sequences of the peptides and a map of the vector are shown below, see FIG. 1 and FIG. 2:

GnRH peptide coding sequence
CTGCAGCAACATTGGAGCTACGGCTTGCGCCCGGGCGCGGTACC
GnRH amino acid sequence
LeuGlnGlnHisTrpSerTyrGlyLeuArgProGlyAlaVal
Neurotensin peptide coding sequence
CTGCAGGAATTGTACGAAAACAAACCGCGCCGCCCGTACATTTTGGCGGT
ACC
Neurotensin peptide
LeuGlnGluLeuTyrGluAsnLysProArgArgProTyrIleLeuAla
Val

The sequenced plasmid is then recombined into the Bacvector-1000 triple cut baculovirus DNA (Novagen) by co-transfection into sf21 cells. The resulting baculoviruses are only viable if recombination has occurred, and are diploid for the gp64 gene, as insertion does not occur in the native gp64 locus. This is essential to preserve high infectivity of the baculovirus, and has been observed in other systems e.g. HIV, where env protein modification can be carried out.

A further modification of the pBACsurf vector was carried out, in order to facilitate a single recombination step for both of the humanising sequences (i.e. human promoter and cell surface attachment), whereby a second multiple cloning site (MCS2) was inserted into the recombination area, which contains unique (i.e. single cut for the plasmid) RE sites. This is shown below:

The alternative method of deriving the multiple recombinants is to co-transfect the promoter vector pBACMAM2 with the singly modified pBACsurf with the Bacvector 1000 triple cut DNA into sf21 insect cells, and to screen for double recombinant viruses by polymerase chain reaction. This is the method of choice when large (>3 kb) promoter fragments are inserted, as the capacity of the pBACsurf (MCS2) vector is limited. Viral DNA from the recombinant plaques therefore is characterised by a wild-type PCR product and a larger product from the insertion recombinant. The sense of the insertion is verified by direct DNA sequencing of the purified PCR product.

Promoter fragments are inserted into the pBACMAM vector to replace the hybrid CAG promoter (CMV enhancer (within Genbank acc.#: AF477200)), Chicken beta actin promoter (Genbank acc.#: E02199) and rabbit beta globin terminator (Genbank acc.#: AX451706). To facilitate this a general insertion construct was prepared in pT7 blue vector, such that the promoter is inserted upstream of either indicator genes (for activity in human cells such as the enhanced green fluorescent protein (EGFP) (Genbank acc.#: U57609) or a hybrid consisting of the EGFP fused to the common bacterial indicator chloramphenicol acetyl transferase or CAT gene (Genbank acc.#: D14641). This construct is then excised from the pT7 blue carrier and inserted via SphI/SwaI and HindIII/BclI sites into the pBACMAM vector. The use of the multiple cloning site in the pBACMAM vector (thus retaining the BglII, StuI, Sae8387, NotI, KpnI, SmaI, Bsu36 and MacI sites) and inserting the promoter construct upstream of the Rabbit beta globin terminator (Genbank acc.#: AX451706) is also possible.

TABLE 1

| Promoter sequence | DNA Accession number |
|---|---|
| Prostate androgen | BC026274 or NM005551 |
| regulated transcript 1 | |
| Prostate transglutaminase, | BC007003 |
| Prostase | XM031805 |
| Prostate-derived Ets factor | AF071538 |
| Prostatic acid phosphatase | X53605 |
| Pr LeuZip | |
| PAGE-4 | AF275258 |
| DD3 | |
| NKX3.1 | AF247704 |
| probasin | AX259949 |
| prostate-specific antigen | AJ459782 |
| prostate-specific | XM165392 |
| membrane antigen | |
| prostate stem cell antigen | XM030742 |
| prostate carcinoma tumour | NM006499 |
| antigen-1 | |
| AIPC | AF338650 |
| Trp-p8 | AC005538 |
| E2F4 | AF527540 |
| Daxx | AF015956 |
| TRPM-2 | NM001831 |
| PART-1 | nm016590 |
| TMPRSS2 | |
| Bomesin | |
| Steap | Nm 012449 |
| TARP | Af151103 |
| PcGEM1 | Af223389 |

TABLE 2

| Tumour suppressor | |
|---|---|
| Polypeptide | DNA accession number |
| p53 | AF136270 |
| Retinoblastoma | |
| APC polypeptide | NM000038 |
| DPC-4 polypeptide | U73825 |
| BRCA-1 polypeptide | |
| BRCA-2 polypeptide | |
| WT-1 polypeptide | XM_034418 |
| MMAC-1 polypeptide | XM083839 |
| Familial polyposis coli | NM000038 |
| polypeptide | |

TABLE 3

| Tumour Rejection | |
|---|---|
| Antigen Precursor Family | DNA Accesssion Number |
| MAGE | XM066465 |
| BAGE | NM001187 |
| GAGE | NM_003785 |
| DAGE | Q99958 |

TABLE 4

| Cell-CycleArrest | |
|---|---|
| Polypeptide | DNA accession number |
| p21 | NM078467 |
| p16 | NM058196 |
| p15 | BC002010 |
| p18 | BC027026 |
| p19 | NM079421 |
| PTEN | AF143312 |

TABLE 5

| Cytokine | DNA Accession number |
|---|---|
| growth hormone | |
| leptin | |
| erythropoietin | |
| prolactin | |
| IL-2 | XM_035511 |
| IL-3 | U81493 |
| IL-4 | AF395008 |
| IL-5 | AF353265 |
| IL-6 | AF039224 |
| IL-7 | NM000880 |
| IL-9 | AF361105 |
| IL-10 | BC022315 |
| IL-11 | BC012506 |
| the p35 subunit of IL-12 | AF101062 |
| IL-13 | AF377331 |
| IL-15 | AF031167 |
| G-CSF | E09569 |
| GM-CSF | M13207 |
| CNTF | E09734 |
| CT-1 | XM096076 |
| LIF | XM009915 |
| oncostatin M | NM020530 |
| IFNα | J00207 |

TABLE 6

| Apoptosis inducing | |
|---|---|
| polypeptide | DNA Accession number |
| P53 | AF136270 |
| adenoviras E3.11.6K | |
| adenoviras E4 | |
| adenovirus f4 | |
| caspase | |
| Fas ligand | E11157 |
| C-Cam 1 | XM113980 |
| ODC | NM052998 |
| OAZ | XM037830 |
| spermidine/spermine N1- | BC002503 |
| acetyltransferase | |
| ZNF145 | NM006006 |
| PTEN phosphatase | AF143312 |
| androgen receptor | NM_000044 |
| Bcl2 family members. | |

TABLE 7

| Prodrug Activating | |
|---|---|
| polypeptide | DNA Accession number |
| cytosine deaminase | AL627278 |
| thymidine kinase | AB078742 |
| nitroreductase RdxA | AY063488 |
| Cytochrome P450 | NM_000761 |
| CYP1A2 | |
| CYP2E1 | AB052259 |
| CYP3A4 | AF209389 |

1. A baculovirus wherein the genome of the virus has been modified to include
i) a nucleic acid molecule that encodes a therapeutic agent;
ii) a nucleic acid molecule that encodes a polypeptide that functions to specifically target the baculovirus to at least one cell type; wherein the baculovirus genome is further modified by addition, deletion or substitution of at least one nucleotide base in a part of a baculovirus gene that encodes an amino acid motif that binds heparin sulphate expressed by a cell.

2. A baculovirus according to claim 1 wherein said motif is present in baculovirus gene gp64.

3. A baculovirus according to claim 2 wherein said amino acid motif comprises the amino acid sequence, hvrk (SEQ ID NO: 7).

4. A baculovirus according to claim 3 wherein said amino acid motif comprises the amino acid sequence, kfnrcikrkvehrvkkrpptwrhnvrak (SEQ ID NO: 1).

5. A baculovirus according to claim 1, wherein said baculovirus genome is adapted for eukaryotic gene expression of said nucleic acid molecules.

6. A baculovirus according to claim 5 wherein said eukaryotic expression is through the provision of cancer cell specific promoter elements.

7. A baculovirus according to claim 6 wherein said promoters are active in prostate cancer cells.

8. A baculovirus according to claim 1, wherein said promoters are selected from the group as represented in Table 1.

9. A baculovirus according to claim 1, wherein said therapeutic agent is a polypeptide.

10. A baculovirus according to claim 9 wherein said polypeptide is a tumour suppressor polypeptide selected from the following group represented in Table 2.

11. A baculovirus according to claim 9 wherein said polypeptide is an antigenic polypeptide.

12. A baculovirus according to claim 9 wherein said polypeptide is a tumour rejection antigen precursor selected from the following polypeptides represented in Table 3.

13. A baculovirus according to claim 9 wherein said polypeptide is a prostate tumour rejection antigen.

14. A baculovirus according to claim 9 wherein said polypeptide is a cytotoxic polypeptide.

15. A baculovirus according to claim 9 wherein said polypeptide is a polypeptide which induces cell-cycle arrest.

16. A baculovirus according to claim 15 wherein said cell-cycle arrest polypeptide is selected from the group represented in Table 4.

17. A baculovirus according to claim 9 wherein said polypeptide is a cytokine.

18. A baculovirus according to claim 17 wherein said cytokine is selected from the group represented in Table 5.

19. A baculovirus according to claim 9 wherein said polypeptide is an antibody, or active binding fragment thereof.

20. A baculovirus according to claim 19 wherein said antibody fragment is a single chain antibody variable region fragment.

21. A baculovirus according to claim 9 wherein said polypeptide is a polypeptide which induces apoptosis.

22. A baculovirus according to claim 21 wherein said apoptosis inducing polypeptide is represented in Table 6.

23. A baculovirus according to claim 9 wherein said polypeptide is a pro-drug activating polypeptide.

24. A baculovirus according to claim 23 wherein said prodrug activating polypeptide is represented in Table 7.

25. A baculovirus according to claim 9 wherein said polypeptide has anti-angiogenic activity.

26. A baculovirus according to claim 1, wherein said therapeutic agent is an antisense nucleic acid molecule.

27. A baculovirus according to claim 1, wherein said therapeutic agent is a double stranded RNA molecule.

28. A baculovirus according to claim 1, wherein said therapeutic agent is a ribozyme.

29. A baculovirus according to claim 1, wherein said baculovirus binds the cell surface by a cell surface receptor expressed by said cell.

30. A baculovirus according to claim 29 wherein said nucleic acid encodes a polypeptide selected from the following group: GnRH (Genbank acc. no: L03380), fibroblast growth factors; insulin and insulin-like growth factors; neurotensin platelet derived growth factor (Genbank acc. no: NM_{—}002609 & NM_{—}006206); somatostatin (Genbank acc. no: BC032625).

31. A baculovirus according to claim 29 wherein said nucleic acid that encodes said polypeptide is inserted into the baculovirus genome at a site which fuses said polypeptide to a baculovirus capsid polypeptide.

32. A baculovirus according to claim 31 wherein the capsid polypeptide is gp64.

33. A composition comprising the baculovirus according to claim 1 and a pharmaceutically acceptable carrier.

34. A composition according to claim 33 wherein the composition further comprises a complement inhibitor.

35. A composition according to claim 34 wherein said complement inhibitor comprises the amino acid sequence ICVVQDWGHHRCT-NH₂ (SEQ ID NO: 2).

36. A composition according to claim 35 wherein said complement inhibitor consists of the amino acid sequence ICVVQDWGHHRCT-NH₂ (SEQ ID NO: 2).

37. A composition according to claim 34 wherein said complement inhibitor is a variant peptide comprising the amino acid sequence ICVVQDWGHHRCT-NH₂ (SEQ ID NO: 2) wherein said sequence is modified by addition, deletion or substitution of at least one amino acid residue and further wherein said inhibitor has improved inhibitory activity with respect to C3 complement protein.

38. (canceled)

39. (canceled)

40. A composition or medicament according to claim 33, wherein said composition further comprises at least one further therapeutic agent.

41. A composition according to claim 40 wherein said therapeutic agent is a chemotherapeutic agent.

42. A method of treatment comprising administering to a subject a therapeutically effective amount of the baculovirus, composition or medicament according to claim 1.

43. A method according to claim 38 wherein said treatment is cancer.

44. A method according to claim 39 wherein said cancer is prostate cancer.

45. A method according to claim 42 wherein said baculovirus, composition or medicament is administered intravenously.
